(19) United States
(12) Patent Application Publication
(10) Pub. No.: US 2018/0001102 A1
(43) Pub. Date: Jan. 4, 2018

(54) PULSED ELECTROMAGNETIC FIELD TREATMENT

(71) Applicant: Optibio Limited, London (GB)

(72) Inventors: William J. Henry, Buckinghamshire (GB); Richard Wolf-Garraway, London (GB)

(21) Appl. No.: 15/702,600

(22) Filed: Sep. 12, 2017

Related U.S. Application Data
(63) Continuation of application No. PCT/EP2016/055383, filed on Mar. 11, 2016.

(30) Foreign Application Priority Data
Mar. 13, 2015 (GB) 1504328.4

(51) Int. Cl. A61N 1/40; A61N 1/02; A61N 1/06; A61N 1/36

(52) U.S. Cl. CPC A61N 1/40; A61N 1/36014; A61N 1/025; A61N 1/06

(57) ABSTRACT

A method of treating a subject includes providing a mobile telecommunications device including a processor, a transceiver coupled to the processor including a transmitter for generating pulsed electrical signals adapted to be coupled to an antenna, at least one memory device accessible by the processor. The mobile telecommunications device is positioned proximate to the subject. Pulsed electrical signals are begun to be generated to cause the transmitter to drive the antenna, wherein the antenna in response to the pulsed electrical signals emits a pulsed electromagnetic field (PEMF) that reaches the subject to provide treatment.

### CROSS REFERENCE TO RELATED APPLICATIONS

This application is a bypass continuation application under 35 U.S.C. 111(a) of International (PCT) application number PCT/EP2016/055383 entitled “PULSED ELECTROMAGNETIC FIELD” that has an international filing date of Mar. 11, 2016 which claims priority to GB application number 1504328.4 filed on Mar. 13, 2015, both of which are incorporated herein in their entireties.

### FIELD

This disclosure relates to a method of treating a subject using pulsed electromagnetic fields, and a method of configuring or reconfiguring a mobile telecommunications device to emit pulsed electromagnetic fields.

### BACKGROUND

Pulsed electromagnetic fields “PEMF” therapy is established in the treatment of a wide spectrum of maladies, disease and conditions. Some devices that deliver PEMF operate in the radio frequency range and these have been proven to benefit a range of conditions.

Every living cell exports positive ions such as sodium and potassium to create an excess of positive charge on the outside of a cell. Therefore, a potential difference across the cell membrane (transmembrane PD) exists. Typically, this potential difference is about 40 mV to say 90 mV, depending on cell type. Like all charged surfaces cell membranes will respond to a modulating EM field by small movements. This enlivens surface receptors and signalling systems that stimulate a cell to function more actively. The signalling systems in the membrane are provoked into stimulating cell activity by the movement. In the case of fibroblasts, for example, the function of this activity is the production of collagen. It is important however that the membrane is allowed to return to its resting position and therefore the EM fields are pulsed. Pulsed Radio Wave Therapy devices are currently available as stand-alone dedicated devices which have a range of settings to provide the optimum pulse radio frequency signal, and come at range of high costs, generally from 350 to 6,000. These devices commonly use electrode-like coils that are used in contact with the body to deliver the PEMF.

Such devices commonly utilise dedicated remote controls or include the software and controls, screens etc. on board the device, increasing cost and reducing flexibility and the potential to upgrade programmes Such devices are sold at a high price as after a sale, manufacturers are limited to an income stream supplying low-cost, genericisable electrodes, gels, test strips, etc. This raises the barrier to purchase and provides a lumpy income stream for manufacturers.

### SUMMARY

This Summary is provided to introduce a brief selection of disclosed concepts in a simplified form that are further described below in the Detailed Description including the drawings provided. This Summary is not intended to limit the claimed subject matter's scope.

Aspects of disclosed embodiments are defined in the appended claims. The Inventors have identified that a smartphone or tablet, for example, with mobile telecommunications capability may be utilised, or its function reconfigured, generally by software alone or by some added hardware, to deliver PEMF, both contact or non-contact, at therapeutic levels. The use of a smartphone, for example, for therapy is believed to be counterintuitive because the use of mobile phones is generally considered to be harmful, e.g., linked to local oedema, haematoma and even brain cancer. This is due to the continuous wave nature of a radio frequency signal for telecommunications. In contrast, the present disclosure relates to the use of pulsed radio waves for patient therapy.

In embodiments, further accessories enhance or supply the PEMF generated by the mobile device. For example, in other embodiments, the combined use of such devices with therapeutic gels provides a synergistic effect. Embodiments take advantage of the connected nature of the mobile device to download different therapeutic programmes and to take micro payments for the use of the program. Embodiments use the connected nature of the device to enable subject details, records of usage, results, progress etc. to be stored both on the device and in the cloud for consultation with a therapist/physician.

This disclosure provides:
non-contact Pulsed Radio Frequency device;
PEMF therapy;
control of electronic therapeutic and diagnostic devices; and
eGels.

The inventors have recognised that a mobile communications device, such as a portable mobile communications device or cellular device or tablet, may be configured or reconfigured to provide functionality which is otherwise only provided by dedicated devices. In particular, the inventors have recognised that telecommunications antenna of mobile telecommunications devices may be driven for use in a method of treatment of the human body or animal, rather than just for telecommunications.

Disclosed embodiments include a method of treating an individual including providing a mobile telecommunications device including a processor, a transceiver coupled to the processor including a transmitter for generating pulsed electrical signals adapted to be coupled to an antenna, at least one memory device accessible by the processor. The mobile telecommunications device is positioned proximate to the subject. Pulsed electrical signals are generated to cause the transmitter to drive the antenna, and the antenna in response to the pulsed electrical signals emits a PEMF that reaches the subject to provide treatment.

### BRIEF DESCRIPTION OF DRAWINGS

Embodiments of the present disclosure will now be described with reference to the accompanying drawings in which:

FIG. 1 shows an example mobile telecommunications device for emitting PEMF for providing treatment to a subject;

FIGS. 2a and 2b show PEMF electrodes connected via the earphone jack of a mobile device;

FIGS. 3a and 3b shows PEMF electrodes connected via an input/output port of a mobile device;

FIG. 4 shows PEMF electrodes connected & controlled wirelessly (BLUETOOTH/WiFi/NFC) via intermediary controller/power source;

FIG. 5 shows PEMF electrodes (which may or may not be connected themselves) connected and controlled directly via wireless technology;

FIG. 6 shows PEMF electrodes connected and controlled via wireless technology and built into device intended to e.g. be laid on, wrapped around head, placed under pillow, etc.;

FIG. 7 shows PEMF electrodes connected and controlled via wireless technology and built into wearable device; and

FIG. 8 shows a chart displaying relative profilometry data wherein a mobile telecommunications app was used to deliver a PEMF to participants.

In the figures, like reference numerals refer to like parts.

### DETAILED DESCRIPTION

Example embodiments are described with reference to the drawings, wherein like reference numerals are used to designate similar or equivalent elements. Illustrated ordering of acts or events should not be considered as limiting, as some acts or events may occur in different order and/or concurrently with other acts or events. Furthermore, some illustrated acts or events may not be required to implement a methodology in accordance with this disclosure.

Also, the terms “coupled to” or “couples with” (and the like) as used herein without further qualification are intended to describe either an indirect or direct electrical connection. Thus, if a first device “couples” to a second device, that connection can be through a direct electrical connection where there are only parasitics in the pathway, or through an indirect electrical connection via intervening items including other devices and connections. For indirect coupling, the intervening item generally does not modify the information of a signal but may adjust its current level, voltage level, and/or power level. The terms antenna and electrode are used interchangeably herein to refer to a direct or indirect transmitter of PEMF.

In overview, this disclosure relates to converting a personal radio device such as a smart phone into pulsed radio wave therapy devices. Disclosed embodiments include a mobile telecommunications device configured for use in a method of treating the human body, but it may be appreciated that this disclosure is equally applicable to the treatment of an animal body. Embodiments disclosed comprise two PEMF electrodes by way of non-limiting example only. The present disclosure extends to any number of PEMF electrodes.

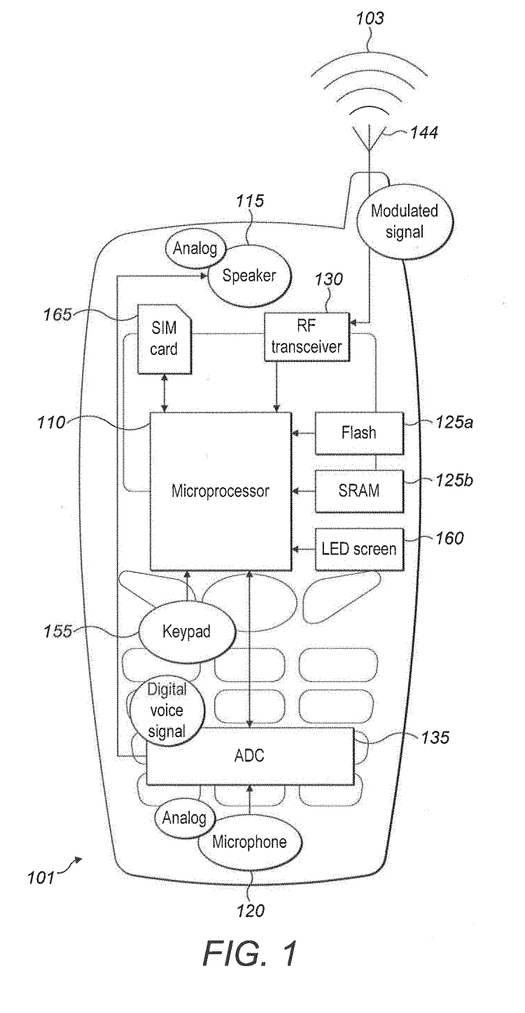

A first embodiment is shown in FIG. 1. FIG. 1 shows an example mobile telecommunications device 101 arranged to emit a PEMF. The mobile telecommunications devices is shown comprising a processor 110 (shown as a microprocessor), a speaker 115 and a microphone 120 coupled by an analog to digital converter (ADC) 135 to the processor 110, at least one memory shown as flash memory 125a and SRAM 125b that are both accessible by the processor 110. A RF transceiver 130 is coupled to the processor 110 and includes a receiver, and a transmitter for generating pulsed electrical signals, both coupled to an antenna 144, where the transmitter is configured to emit a PEMF 103 for use in a method of treating an individual (e.g., the human body). The mobile telecommunications device 101 is also shown including a keypad 155, LED screen, and a subscriber identification module (SIM) card 165.

A commercially available mobile telecommunications device (smartphone) can be modified to emit a PEMF. Such a commercially available mobile telecommunications device (smartphone) is configured to receive and emit carrier (sine) waves such as GSM, WI-FI, NFC and Bluetooth. These carrier waves are typically used to carry content such as sound or video data.

Software can be used to control existing hardware of the mobile telecommunications device in such a way that the carrier sine waves are pulsed (i.e. turned on and off) such that the periods of activity (ON periods) and inactivity (OFF periods) provide a cycle of activities that have been found to have a therapeutic effect.

This results in the transformation of high frequency non-pulsed waves, into targeted pulses of low frequency square waves, which the body perceive in line with a range of electrical frequencies commonly found in, or created by the systems of the subject, such as the human body.

Disclosed embodiments can use a smartphone to pulse the carrier wave at specified frequencies so as to produce square waves at the specified frequencies. This creates a desired functional (or working) wave by using a higher frequency carrier wave. To do this the smartphone is programmed so as to turn the carrier wave on and off at the desired functional frequencies. In this way for example a smartphone that emits for example BLUETOOTH at a carrier frequency of 2.4 GHz can be used to produce functional waves at a pulse frequency of between 1 Hz and 300 Hz which are more useful to humans and other animals, particularly in the range of between 3 Hz and 100 Hz. As known in the art of communications the BLUETOOTH protocol is a standardized protocol for sending and receiving data currently via a 2.4 GHz wireless link that utilizes a carrier frequency in a band from 2.4 GHz to 2.483 GHz. Without this method a commercially available smartphone cannot produce functional waves (PEMF) at these low pulse frequencies. Furthermore the modification can provide for the rapid change from one functional frequency to another many times during a duty cycle.

In some embodiments, the mobile telecommunications device is arranged for wireless telecommunication with other mobile telecommunications devices. In embodiments, the mobile telecommunications device is a mobile telephony device. However, it will be appreciated that the present disclosure extends to the modification of any mobile telecommunications device.

In some embodiments, the PEMF is configured to interact with the human body. A method of treating a subject can comprise positioning the mobile telecommunications device proximate to the subject, beginning generating pulsed electrical signals to cause the transmitter to drive the antenna, and the antenna in response emits a pulsed electromagnetic field that reaches the subject. The subject as used herein can refer to a human being or an animal such as a dog or a cat. As used herein, the mobile telecommunications device being “proximate to the subject” generally refers to a distance less than 2 meters, generally less than a meter that can include direct physical contact. The PEMF can be at a carrier frequency between 300 MHz and 6 GHz, and emitted as a series of pulses at a pulse frequency 1 to 300 Hz, such as at 3 to 100 Hz.

In embodiments, the PEMF has at least one parameter selected to enhance the interaction of the PEMF with the human body. Such parameters include functional wave frequency, changes of functional wave frequency, pulse width (time), pulse rest width (time), duty cycle, and power (which is a function of the selected carrier frequency and the duty cycle). The power of the PEMF emitted by the mobile telecommunications device 101 is generally in the range of 0.25 mW to 100 mW, and more usually in the range of 0.5 mW to 5 mW, and most commonly currently in the range 2mW to 3mW when emitting BLUETOOTH but may be between 0.5 W and 2.5 W (or most common currently between 1 W and 2 W) when emitting in the GSM frequency band. In embodiments, the carrier wave has a frequency in the GSM frequency band which is currently generally about 380 MHz to 1900 MHz.

In embodiments, the carrier wave has a frequency of 300 to 3000 MHz (3 GHz), optionally, 2300-2500 MHz, further optionally, 2400-2483.5 MHz which corresponds to the current BLUETOOTH protocol standard.

In embodiments, the PEMF is emitted in 5-15 minute bursts separated by rest periods of 1-10 minutes, optionally 9-11 minute bursts separated by rest periods of 4-6 minutes. In other embodiments, the PEMF is emitted in 1 to 120 second bursts separated by rest periods of 1 to 120 seconds. In embodiments, the PEMF is emitted in pulses at a pulse frequency of 1 to 300 Hz, optionally 1 to 40 Hz, further optionally 3 to 13 Hz, further optionally, 1 to 20 Hz. In embodiments, the pulse bursts are emitted for a total time duration of 1 to 12 hours, such as 0.5-4 hours, 1.5-2.5 hours, or 3 to 9 hours.

In embodiments the mobile telecommunications device is arranged to vary the pulse frequency of the PEMF during treatment. It may be understood that the PEMF is emitted at a first pulse frequency for a first time period, followed by a second pulse frequency for a second time period, wherein the first pulse frequency is different from the second pulse frequency. In embodiments the first pulse frequency is lower than the second pulse frequency. In other embodiments, the first pulse frequency is higher than the second pulse frequency.

In the embodiment shown in FIG. 1, the antenna 144 is an internal antenna of the mobile telecommunications device 101. In embodiments, the antenna is a radio-frequency mobile telecommunications antenna of the mobile telecommunications device. In embodiments the antenna is a BLUETOOTH™ antenna. In embodiments the antenna is a WiFi antenna. In embodiments the antenna is a near field communications (NFC) antenna which is known in the art to comprise a ferrite antenna including a primary antenna coil wound on a ferrite core of the ferrite antenna, and a loop coil provided on a side of the ferrite antenna in a position where the loop coil is interlinked with magnetic flux generated by the ferrite antenna.

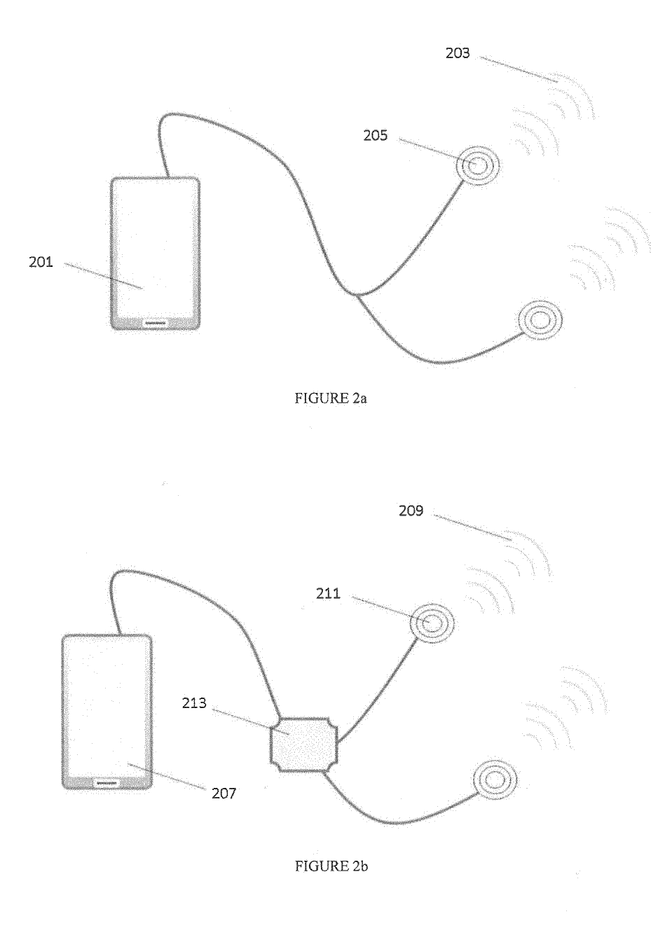

A further embodiment is shown in FIGS. 2a and 2b.

FIG. 2a shows an example mobile telecommunications device 201 generally having the same components as mobile telecommunications device 101 shown in FIG. 1 connected to two PEMF electrodes 205 via the earphone jack of the mobile communications device 201. The PEMF electrodes 205 are driven to emit the PEMF 203.

FIG. 2b shows a mobile telecommunications device 207 connected to two PEMF electrodes 211 via the earphone jack of the mobile communications device 207 and via an intermediary device 213. The PEMF electrodes 211 are driven to emit the PEMF 209.

In embodiments, it may therefore be understood that the antenna is an external antenna coupled to the mobile telecommunications device. In embodiments, the external antenna is wired to the mobile telecommunications device. In the embodiments, the external antenna is wired to a headphone or microphone jack of the mobile telecommunications device.

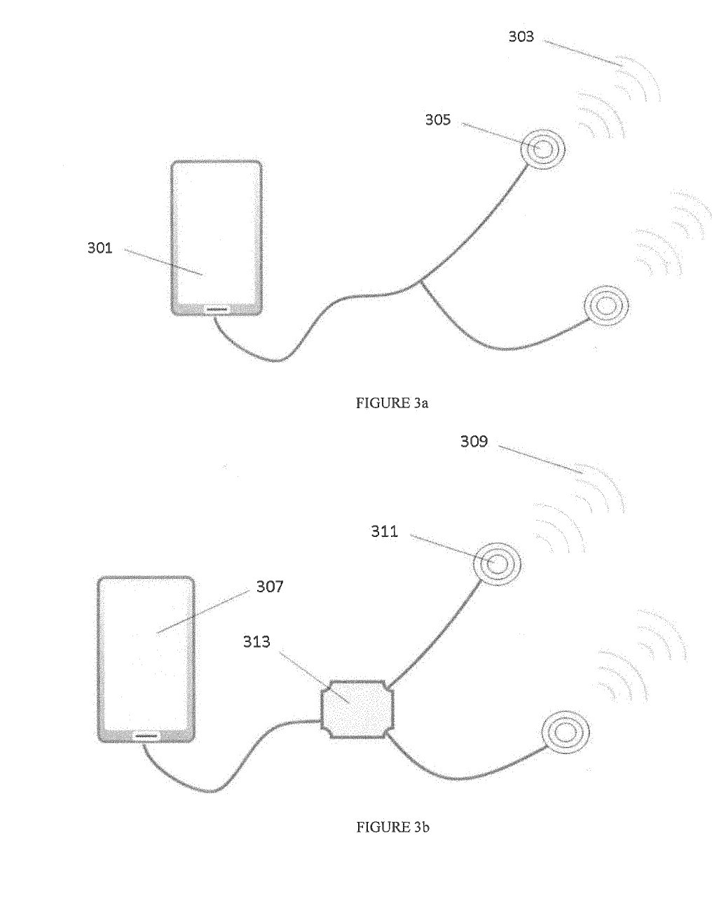

Embodiments are shown in FIGS. 3a and 3b.

FIG. 3a shows a mobile telecommunications device 301 generally having the same components as mobile telecommunications device 101 shown in FIG. 1 connected to two PEMF electrodes 305 via an input/output, “I/O”, port of the mobile communications device 301. The PEMF electrodes 305 are driven to emit the PEMF 303.

FIG. 3b shows a mobile telecommunications device 307 generally having the same components as mobile telecommunications device 101 shown in FIG. 1 connected to two PEMF electrodes 311 via an input/output, “I/O”, port of the mobile communications device 307 and via an intermediary device 313. The PEMF electrodes 311 are driven to emit the PEMF 309.

It may therefore be understood that, in embodiments, the external antenna is wired to an input-output port of the mobile telecommunications device.

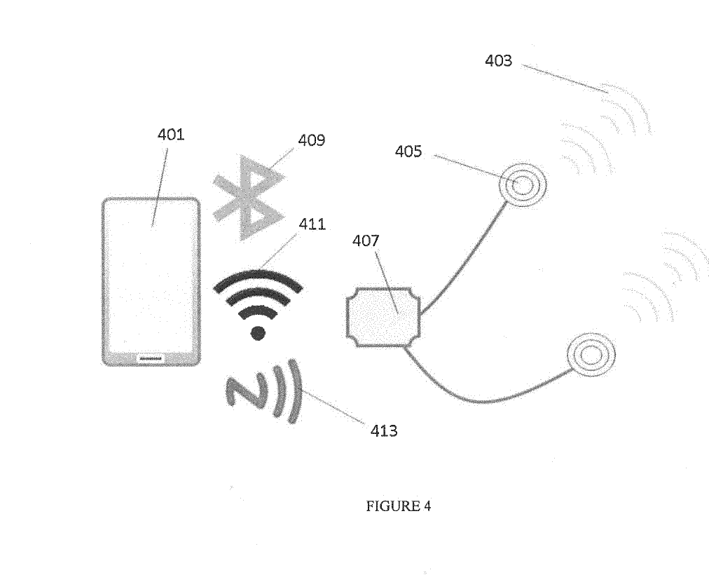

An embodiment is shown in FIG. 4.

FIG. 4 shows a mobile telecommunications device 401 generally having the same components as mobile telecommunications device 101 shown in FIG. 1 wirelessly-connected to two PEMF electrodes 405 via an intermediary controller or power source 407. The PEMF electrodes 405 are driven to emit the PEMF 403. In embodiments, the wireless-connection utilises the BLUETOOTH 409, WiFi 411 or NFC 413 protocol.

It may therefore be understood that in embodiments, the external antenna is wirelessly-coupled to the mobile telecommunications device. In embodiments, the external antenna is wirelessly-coupled to the mobile telecommunications device by BLUETOOTH, WiFi or NFC. In embodiments, the external antenna further comprises an intermediary controller or an intermediary power source.

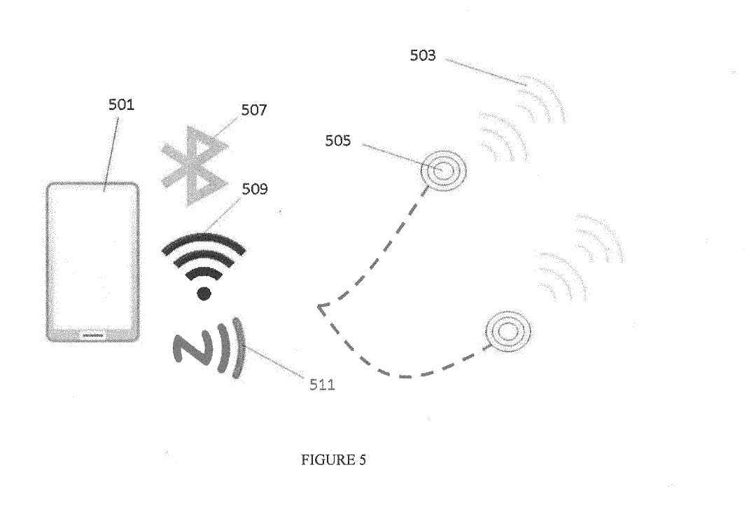

A further embodiment is shown in FIG. 5. FIG. 5 shows a mobile telecommunications device 501 generally having the same components as mobile telecommunications device 101 shown in FIG. 1 wirelessly-connected to two PEMF electrodes 505. The PEMF electrodes 505 are driven to emit the PEMF 503. The wireless-connection utilises the BLUETOOTH 507, WiFi 509 or NFC 511 protocol. In this embodiment, the PEMF electrodes 505 are connected and controlled directly via wireless technology. The PEMF electrodes 505 may or may not be connected themselves.

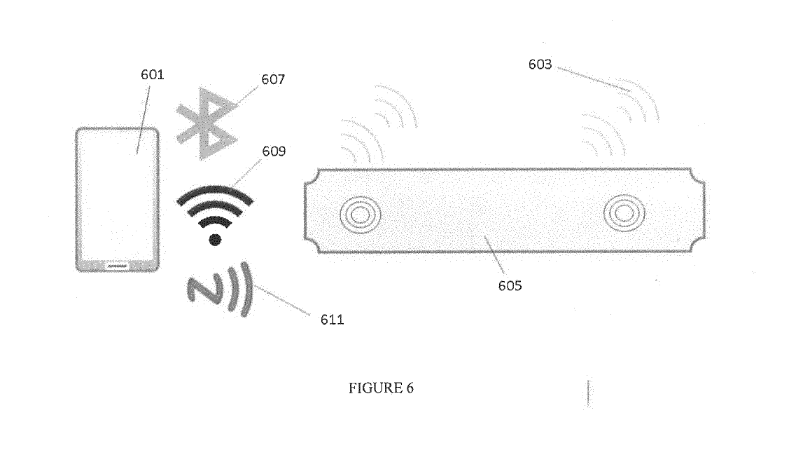

Another embodiment is shown in FIG. 6. FIG. 6 shows a mobile telecommunications device 601 generally having the same components as mobile telecommunications device 101 shown in FIG. 1 wirelessly-connected to a device 605 intended to be laid on, wrapped around the head and/or placed under a pillow, for example The wireless-connection utilises the BLUETOOTH 607, WiFi 609 or NFC 611 protocol. PEMF electrodes are built into the device 605 and are driven to emit the PEMF 603. The electrodes are therefore connected and controlled via wireless technology.

It may be understood that there is therefore provided a system for use in a method of treating the human body, the system comprising: the mobile telecommunications device as per the earlier embodiments; and a peripheral device arranged to house the external antenna.

In embodiments, the peripheral device is a device arranged to receive the human body. In embodiments, the device is a device arranged to be laid on, a device arranged to wrap around the head or a device arranged to be placed under a pillow.

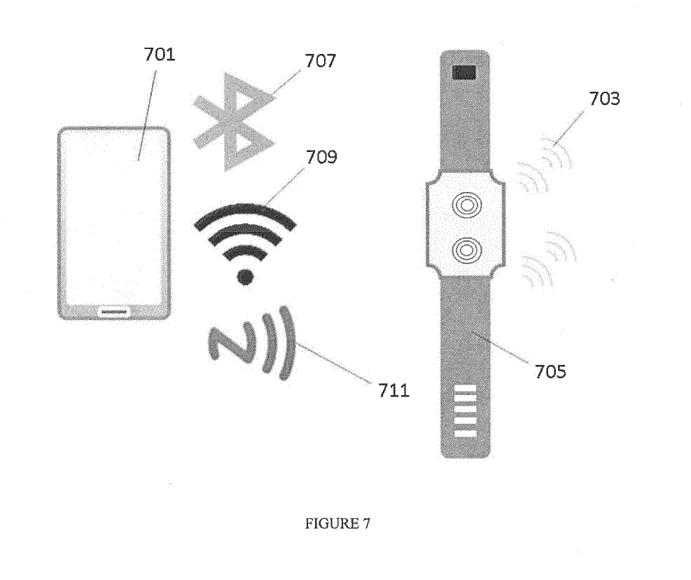

A yet further embodiment is shown in FIG. 7.

FIG. 7 shows a mobile telecommunications device 701 generally having the same components as mobile telecommunications device 101 shown in FIG. 1 wirelessly-connected to a wearable device 705. The wireless-connection utilises the BLUETOOTH 707, WiFi 709 or NFC 711 protocol. PEMF electrodes are built into the wearable device 705 and are driven to emit the PEMF 703. The electrodes are therefore connected and controlled via wireless technology.

In embodiments, the peripheral device is a wearable device such as a watch. In embodiments, the device is used with a gel. In embodiments, the system further comprises a gel configured for use in the method of treating the human body. In embodiments, the gel is configured to improve coupling of the PEMF into the human body.

In embodiments, the PEMF treatments are used in conjunction with a generic electrode gel to provide good contact. In embodiments, these gels convey no therapeutic benefit.

Embodiments use a gel, from a range of possible gels, which work synergistically with the PEMF devices (contact or non-contact). A gel can be applied to the subject. The gel material is selected so that effect of the gels may be enhanced by the use of PEMF or the gels may convey/amplify the current/field of the device or a combination of the two.

In embodiments, there is provided a unique combination of a particular gel with a particular device, or an application setting on a particular device (e.g. “bone healing”, “tendon repair” etc.). In embodiments, the combination of gel and device/application is approved together as a treatment (cf. a pharmaceutical plus a particular delivery device).

In embodiments, the PEMF and gel are arranged to act upon at least one biological cell to provide a therapeutic effect, optionally a synergistic therapeutic effect. In embodiments, the at least one biological cell is a plurality of biological cells. In embodiments, the at least one biological cell is a human cell or an animal cell.

In embodiments, the PEMF is arranged to stimulate the at least one biological cell to initiate production of a substance and the gel is configured to enhance production of the substance.

In embodiments, the gel is configured to stimulate the at least one biological cell to initiate production of a substance and the PEMF is arranged to enhance production of the substance.

In embodiments, the substance is collagen. In embodiments, the gel is configured to supply nutrients to the at least one biological cell and the PEMF is arranged to enhance absorption of the nutrients into the at least one biological cell.

In embodiments, the substance is a peptide, optionally Palmitoyl tetrapeptide 7, and Palmitoyl tripeptide 1.

In embodiments, the gel is configured to supply nutrients to the biological cell(s) and the PEMF is arranged to enhance absorption of the nutrients into the at least one biological cell.

In embodiments, the PEMF is arranged to stimulate at least one biological cell and the gel is arranged to supply nutrients to the stimulated at least one biological cell.

There is provided a method of configuring, or reconfiguring, a mobile telecommunications device to drive an antenna with an electrical signal to emit a PEMF configured for use in a method of treating the human body.

The ordinary skilled person will understand that this configuring or reconfiguring of a mobile telecommunications device may be achieved using any one of a variety of different hardware and software solutions. In embodiments, an additional driver is coupled to the mobile telecommunications device to provide the appropriate signals to a telecommunications antenna. The ordinary skilled person understands how to design an additional driver to provide the appropriate pulsed electrical signals for an antenna. In embodiments, the driver is controllable by an Application installed on the mobile telecommunications device. The ordinary skilled person knows how to provide an Application for driving the additional driver.

The ordinary skilled person will understand that in embodiments it may be necessary to disable a telecommunication function of the device whilst the electrical signal in accordance with embodiments of the present disclosure is provided to the antenna. The ordinary skilled person understands how any necessary switching might be provided to accommodate the driver in accordance with embodiments of the present disclosure.

There is therefore provided a computer program or application arranged to provide instructions to a driver of a mobile telecommunications device to produce an electrical signal configured to drive an antenna to emit a PEMF configured for use in a method of treating the human body.

In an embodiment, the computer program or app is further arranged to receive user-selection of a treatment program from a plurality of treatment programmes wherein the treatment program defines parameters of the electrical pulse signal.

In an embodiment, the computer program or app is further arranged to receive payment from a user for the user-selected treatment program and, optionally, a gel to accompany the treatment program.

In an embodiment, the computer program or app is further arranged to store or upload data related to use of the treatment programmes In an embodiment, the computer program or app is further arranged to store or upload medical data obtained from a user of a treatment programme.

There is provided an installed application or modification to a smart phone or mobile telephone that when operated takes control of the radio frequency transmitter portion of the device to provide pulsed radio waves at a signal strength appropriate to treat a subject within a few meters of the device.

In embodiments, various applications are installed for various therapies that modify the pulse radio-wave profile to suit. In embodiment, the application:
a. provides a selection of therapies to the user and thus tells the control application which program to apply (for example, varying the voltage, current, length of treatment, pulsing of current (time of pulse and time between pulse), etc.);
b. enables the user to purchase and download additional therapy programmes;
c. enables micropayments to be taken, for example:
   i. in-App pay-per use for the programs
   ii. download top up credits to enable the use of program (e.g., pay-as-you-go phones)

In embodiments, the application is designed to arrange micropayments for pay-per-use or top up credits. In embodiments, the application is sold in combination with a gel to accompany and enhance the radio wave therapy effect for use with:
Cosmetic gels
Pain relieving gels
Biolubrication gels
Regenerative gels
Wound healing gels

In embodiments, gels are sold with a credit allocation to be downloaded etc. In embodiments, gels are sold with usage credits, e.g. code in the box, QR code etc. that enables a certain amount of credits to be downloaded, linked to a particular treatment regime in the App., e.g. enough treatments for the expected life of the purchased gel (i.e. 11 treatments if the tube of gel contains 11 applications).

It may be recognised that generally any device with a RF transmitter for generating a radio signal can be modified to provide the device in accordance with the present disclosure. It may also be recognised that the present disclosure extends to exploiting any EM transmitter e.g. WiFi or BLUETOOTH functions.

There is provided an installed application on a mobile device (e.g. tablet or phone) that can control the voltage and current output from either the USB/MHL socket (5V output max., Android and alike) or the headphone/microphone jack (2V output max.).

There is also provided an accessory electrode or electrodes, or intermediate control device that terminates in coils, that plug into the controlled socket to enable delivery of PEMFs to the body of the subject.

In embodiments using the 3.5 mm headphone jack, this may pick up on the “live” microphone contact in the socket, thus the accessory may also retain a pass through headphone jack to enable to user to continue to listen to music etc.

There is further provided an application that provides a selection of therapies to the user and thus tells the control application which program to apply (for example, varying the voltage, current, length of treatment, pulsing of current (time of pulse and time between pulse), etc.).

The application may allow enable the user to purchase and download additional therapy programmes The application may enable micropayments to be taken, for example: (i) in-App pay-per use for the programmes; and (ii) download top up credits to enable the use of programmes (cf. pay-as-you-go phones).

There is provided a range of therapeutic gels that are “tuned” to particular therapy regimes, e.g. joint pain. In an embodiment, the gels are electrically conductive and used on the site of electrode placement but not necessarily. In embodiments, the gels are sold with usage credits, e.g. code in the box, QR code etc. that enables a certain amount of credits to be downloaded, linked to a particular treatment regime in the App., e.g. enough treatments for the expected life of the purchased gel (i.e. 11 treatments if the tube of gel contains 11 applications).

There is yet further provided an application that is installed on a smartphone or tablet that effectively acts as a remote control for a new or existing electronic therapeutic or diagnostic device and that:
a. provides a selection of therapies/diagnostic tests to the user, compatible with the capabilities of the target device and thus tells the control application which program to apply (for example, varying the voltage, current, length of treatment, pulsing of current (time of pulse and time between pulse), etc.);
b. enables the user to purchase and download additional therapy programmes as they are developed;
c. enables micropayments to be taken, for example:
   i. in-App pay-per use for the programs;
   ii. download top up credits to enable the use of programs (cf. pay-as-you-go phones); and
   iii. top up credit vouchers/codes to be supplied with consumables (electrodes, gels, test strips etc.) to enable the device and ensure brand loyalty vs generic versions of consumables;
d. enables a recording of use to be archived/sent to care provider so that:
   i. care provider can verify that a prescribed therapeutic regime has been properly followed
   ii. diagnostic results can be sent to a care provider—alerts could be sent.
      The described methods may be implemented by a computer program. The computer program which may be in the form of a web application or ‘app’ comprises computer-executable instructions or code arranged to instruct or cause a computer or processor to perform one or more functions of the described methods. The computer program may be provided to an apparatus, on a computer readable medium or computer program product. The computer readable medium or computer program product may comprise non-transitory media such as as semiconductor or solid state memory, magnetic tape, a removable computer memory stick or diskette, a random access memory (RAM), a read-only memory (ROM), a rigid magnetic disc, and an optical disk, such as a CD-ROM, CD-R/W, DVD or Blu-ray. The computer readable medium or computer program product may comprise a transmission signal or medium for data transmission, for example for downloading the computer program over the Internet.

An apparatus or device may be configured to perform one or more functions of the described methods. The apparatus or device may comprise a mobile phone, tablet or other mobile processing device. The apparatus or device may take the form of a data processing system. The data processing system may be a distributed system. For example, the data processing system may be distributed across a network or through dedicated local connections. The apparatus or device typically comprises at least one memory for storing the computer-executable instructions and at least one processor for performing the computer-executable instructions.

Although aspects and embodiments have been described above, variations can be made without departing from the inventive concepts disclosed herein. For example, it may be understood that the aspects and embodiments described above are equally suitable for the body of an animal.

### EXAMPLES

Disclosed embodiments of the invention are further illustrated by the following specific Examples, which should not be construed as limiting the scope or content of this Disclosure in any way.

The inventors have found that both the application alone and in combination with an anti-ageing treatment gel reduced physical wrinkles significantly more than a control application over 8 weeks of treatment.

A randomised home-use study in three parallel groups of healthy volunteers with wrinkles was performed in order to assess the efficacy of a mobile device application on a treated site against an untreated site, and a control (inactive app). The study design along with the test groups and protocols are shown in Table 1.

TABLE 1

| Study | | |
|---|---|---|
| design | Double-blind, within-subject comparison, whole face design. | |
| Test | GROUP 1: DH3942 and | DH3942 applied |
| groups | ELECTROMAGNETIC | twice daily |
| and | THERAPY APP | Application used for |
| protocols | (n = 7)* | 6 hours over night |
| | GROUP 2: | Application used for |
| | ELECTROMAGNETIC | 6 hours over night |
| | THERAPY APP | |
| | (n = 5)* | |
| | GROUP 3: | Application used for |
| | ELECTROMAGNETIC | 6 hours over night |
| | THERAPY CONTROL APP | |
| | (n = 5)* | |

For groups 1 and 2 in Table 1, an ‘app’ on a mobile telecommunications device was configured to a control a BLUETOOTH™ transmitter inside the mobile telecommunications device to deliver a PEMF to the participants in each group every night during sleep. The frequency of the electromagnetic field itself was in the BLUETOOTH frequency range, 2.400 to 2.483.5 GHz. The PEMF emission patterns induced by the BLUETOOTH transmitter were as follows:
Pulse at 13 Hz for 10 mins then off for 5 mins, this continues for 2 hours, then
Pulse at 26 Hz for 10 mins then off for 5 mins, this continues for 2 hours, then
Pulse at 39 Hz for 10 mins then off for 5 mins, this continues for 2 hours, then End.

For group 3, a dummy app was used without the knowledge of the participant. A gel, DH3942, was applied to the skin of participants in test group 1. The components of the gel are shown in Table 2.

TABLE 2
DH3942 Composition

Aqua
Phosphatidylcholine
Alcohol
Glycerin
Carbomer
Polysorbate 80
Disodium Phosphate
Benzylalcohol
Methylparaben
Ethylparaben
Sodium Hydroxide
Citric Acid
Linalool
Disodium Edta
Ascorbyl Palmitate
Sodium Metabisulfite
BHT
Palmitoyl Tetrapeptide-7
Palmitoyl Tripeptide-1

In addition to phospholipid vesicles, the three main components of the gel were Palmitoyl ascorbic acid (vitamin C tethered to palmitic acid), Palmitoyl tetrapeptide 7, and Palmitoyl tripeptide 1.

The purpose of vitamin C is to provide a necessary co-factor for the transformation of newly synthesised collagen. Vitamin C is part of the enzyme system that hydroxylates collagen such that it can adopt the correct 3-dimensional structure. The absence of vitamin C would mean that collagen is produced but it can't be adopted into skin structure. Given that an excess of collagen by the skin's fibroblasts is promoted there is a need for excess vitamin C to ensure the fibroblasts are convinced collagen is being degraded by the presence of the peptides.

The two peptides on the other hand promote the synthesis of collagen. In effect when fibroblasts encounter these peptides they are provoked into producing collagen since the peptides represent the breakdown products of collagen. In other words the fibroblasts have a signal that collagen is being degraded and respond by producing more collagen.

It may therefore be understood that the PEMF may stimulate fibroblasts to initiate production of collagen and the gel may enhance production of collagen by the fibroblasts. Conversely, the gel may stimulate the fibroblasts to initiate production of collagen and the PEMF may enhance production of collagen by the fibroblasts. It may also be understood that the gel is configured to supply components of the gel to the fibroblasts and the PEMF is arranged to enhance absorption of the components of the gel into the fibroblasts.

Phospholipids of the base vesicles will also be used during the production of increased sub-dermal structures since all cells require phospholipid as part of their external and internal membranes.

The reduction in wrinkle volume was assessed by profilometry. For these measurements, Silflo replicas were made of the same patch of skin on each subject at each assessment time. The volume of the ridges on these moulds—effectively the volume of the wrinkles on that patch of skin—were then measured in the following manner. A collimated light source directed at a 25° angle from the plane of the replica was used. The sampling orientation was adjusted to assess a combination of the expression-induced lines (crow's feet) and minor, fine lines. The shadow texture produced by the oblique lighting of the negative replica was analysed. Raw data from the profilometry assessments are detailed in Table 3.

The following abbreviations are used in Table 3 and thereafter:
“Comb.”=GROUP 1: DH3942 and electromagnetic therapy app combined;
“App.”=GROUP 2: electromagnetic therapy app alone;
“Control”=GROUP 3: electromagnetic therapy control app (placebo).

TABLE 3

| | PROFILOMETRY | | |
|---|---|---|---|
| | Week | Week | Week |
| Group | 0 | 4 | 8 |
| Comb. | 131 | 125 | 118 |
| | 133 | 127 | 120 |
| | 126 | 121 | 115 |
| | 135 | 128 | 123 |
| | 127 | 122 | 116 |
| | 130 | 124 | 119 |
| | 136 | 131 | 123 |
| MEAN | 131.1 | 125.4 | 119.1 |
| STDEV | 3.8 | 3.5 | 3.1 |
| App | 126 | 122 | 119 |
| | 129 | 125 | 121 |
| | 137 | 131 | 128 |
| | 126 | 122 | 119 |
| | 131 | 126 | 123 |
| MEAN | 129.8 | 125.2 | 122.0 |
| STDEV | 4.5 | 3.7 | 3.7 |
| Control. | 127 | 129 | 130 |
| | 129 | 126 | 127 |
| | 133 | 131 | 130 |
| | 125 | 126 | 127 |
| | 128 | 127 | 129 |
| MEAN | 128.4 | 127.8 | 128.6 |
| STDEV | 3.0 | 2.2 | 1.5 |

For each type of assessment, two further variables were constructed, as detailed in Table 4, namely the difference between Week 0 and Week 4 and 8 and the ratio of Week 4 and 8 to Week 0 (normalisation).

TABLE 4
273

| | PROFILOMETRY | | | | | | |
|---|---|---|---|---|---|---|---|
| | Week | Week | Week | Wk 0 − | Wk 0 − | Wk 4/ | Wk 8/ |
| Group | 0 | 4 | 8 | Wk 4 | Wk 8 | Wk 0 | Wk 0 |
| Comb. | 131 | 125 | 118 | 6.0 | 13.0 | 0.95 | 0.90 |
| | 133 | 127 | 120 | 6.0 | 13.0 | 0.95 | 0.90 |
| | 126 | 121 | 115 | 5.0 | 11.0 | 0.96 | 0.91 |
| | 135 | 128 | 123 | 7.0 | 12.0 | 0.95 | 0.91 |
| | 127 | 122 | 116 | 5.0 | 11.0 | 0.96 | 0.91 |
| | 130 | 124 | 119 | 6.0 | 11.0 | 0.95 | 0.92 |
| | 136 | 131 | 123 | 5.0 | 13.0 | 0.96 | 0.90 |
| MEAN | 131.1 | 125.4 | 119.1 | 5.7 | 12.0 | 0.956 | 0.91 |
| STDEV | 3.8 | 3.5 | 3.1 | 0.8 | 1.0 | 0.01 | 0.01 |
| App. | 126 | 122 | 119 | 4.0 | 7.0 | 0.97 | 0.94 |
| | 129 | 125 | 121 | 4.0 | 8.0 | 0.97 | 0.94 |
| | 137 | 131 | 128 | 6.0 | 9.0 | 0.96 | 0.93 |
| | 126 | 122 | 119 | 4.0 | 7.0 | 0.97 | 0.94 |
| | 131 | 126 | 123 | 5.0 | 8.0 | 0.96 | 0.94 |
| MEAN | 129.8 | 125.2 | 122.0 | 4.6 | 7.8 | 0.965 | 0.94 |
| STDEV | 4.5 | 3.7 | 3.7 | 0.9 | 0.8 | 0.01 | 0.00 |
| Control | 127 | 129 | 130 | −2.0 | −3.0 | 1.02 | 1.02 |
| | 129 | 126 | 127 | 3.0 | 2.0 | 0.98 | 0.98 |
| | 133 | 131 | 130 | 2.0 | 3.0 | 0.98 | 0.98 |
| | 125 | 126 | 127 | −1.0 | −2.0 | 1.01 | 1.02 |
| | 128 | 127 | 129 | 1.0 | −1.0 | 0.99 | 1.01 |
| MEAN | 128.4 | 127.8 | 128.6 | 3.0 | −1.0 | 1.00 | 1.00 |
| STDEV | 3.0 | 2.2 | 1.5 | 2.1 | 2.6 | 0.02 | 0.02 |

The statistical significance of the results in Table 4 is shown in Table 5 for each of the test groups. In Table 5 the following conventions for levels of significance have been used:

TABLE 5

| | | | | Difference from | | Normalised | |
|---|---|---|---|---|---|---|---|
| | Raw data | | | Week 0 | | (=% Wk 0) | |
| | Wk | Wk | Wk | Wk 0 − | Wk 0 − | Wk 4/ | Wk 8/ |
| | 0 | 4 | 8 | Wk 4 | Wk 8 | Wk 0 | Wk 0 |
| Comb. | ns | ns | ns | * | **** | * | **** |
| vs App. | | | | | | | |
| Comb. | ns | ns | *** | ns | **** | ** | *** |
| vs. | | | | | | | |
| Control | | | | | | | |
| App. vs. | ns | ns | ** | ** | ** | ** | ** |
| Control | | | | | | | |

ns p > 0.05;
* p ≦ 0.05;
** p ≦ 0.01;
*** p ≦ 0.001;
**** P ≦ 0.0001

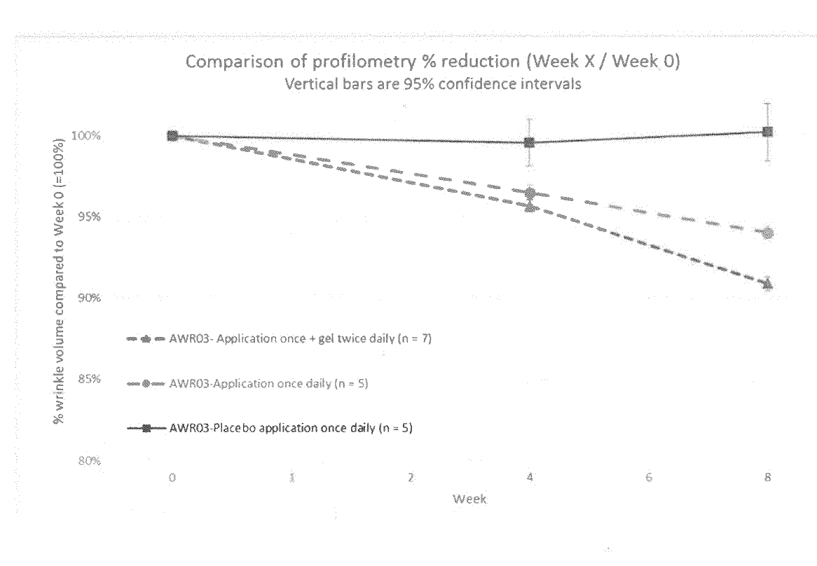

FIG. 8 is a chart comparing reduction in wrinkle volume over 8 weeks as a percentage of wrinkle volume at week 0 (100%). The abscissa displays the number of weeks within a range from 0-8 weeks. The ordinate displays the wrinkle volume as a percentage of the wrinkle volume observed at week 0 within a range from 80-100%. The chart displays the mean data from the last two columns of Table 4.

By week 8, wrinkle volume (raw data) for both the combined treatment and the Application alone were significantly less than the Control application. The actual differences (difference from week zero, i.e. removing any variation in week 0 data) show that at week 4 and 8, the Combination reduced wrinkle volume significantly more than both the Application alone and the Control. The Application was more effective than the Control at week 4 (nearly significantly at p=0.054), and significantly so by week 8.

This was reflected in the normalised data where the reduction achieved by the combination (9.9%) was significantly better than the application alone (6%) which was significantly better than the Control (an increase of 0.2%) at all time points.

The application alone significantly reduces wrinkle volume over the placebo application by week 4. The addition of an anti-wrinkle gel formulation significantly enhances the effect of the application by week 8.

1. A method of treating a subject, comprising:
providing a mobile telecommunications device including a processor, a transceiver coupled to the processor, the transceiver including a transmitter for generating pulsed electrical signals adapted to be coupled to an antenna, the mobile telecommunications device further including at least one memory device accessible by the processor;
positioning the mobile telecommunications device proximate to the subject; and
beginning generating the pulsed electrical signals to cause the transmitter to drive the antenna,
wherein the antenna in response to the pulsed electrical signals emits a pulsed electromagnetic field (PEMF) that reaches the subject.

2. The method of claim 1, wherein the antenna is an internal mobile telecommunications antenna of the mobile telecommunications device.

3. The method of claim 1, wherein the antenna is an external antenna coupled to the transmitter of the mobile telecommunications device.

4. The method of claim 1, wherein a carrier wave for the PEMF is at a frequency between 300 MHz and 6 GHz.

5. The method of claim 1, wherein the PEMF is emitted as a series of pulses at a pulse frequency 1 to 300 Hz.

6. The method of claim 1, further comprising applying a gel to a selected location on said subject.

7. The method of claim 1, wherein the PEMF comprises 1- to 120-second bursts separated by rest periods of 1 to 120 seconds. The method of claim 1, wherein the PEMF comprises 3- to 45-minute bursts separated by rest periods of 1 to 10 minutes.

9. The method of claim 8, wherein the bursts are 9- to 11-minute bursts separated by the rest periods of from 4 to 6 minutes.

10. The method of claim 1, wherein the PEMF is emitted for a total time duration of 1 to 12 hours.

11. The method of claim 1, wherein the PEMF is emitted at a first pulse frequency for a first time period, followed by a second pulse frequency for a second time period, wherein the first pulse frequency is different from the second pulse frequency.

12. The method of claim 1, wherein said mobile telecommunications device is configured to operate using a Bluetooth protocol.

13. The method of claim 4, wherein said carrier wave is at a frequency in a band from 2.4 GHz to 2.483 GHz and a pattern of the PEMF is as follows:
for a first period of two hours, pulses at a pulse frequency of 13 Hz for 10 minutes then off for 5 minutes, then for a second period of two hours, pulses at a pulse frequency of 26 Hz for 10 minutes then off for 5 minutes, then for a third period of two hours, pulses at a pulse frequency of 39 Hz for 10 minutes then off for 5 minutes.

14. A method of treating a subject, comprising:
providing a mobile telecommunications device including a processor, a transceiver coupled to the processor, the transceiver including a transmitter for generating pulsed electrical signals adapted to be coupled to an antenna, the mobile telecommunications device including at least one memory device accessible by the processor;
positioning the mobile telecommunications device proximate to the subject; and
beginning generating the pulsed electrical signals to cause the transmitter to drive the antenna;
wherein the antenna in response to the pulsed electrical signals emits a pulsed electromagnetic field (PEMF) that reaches the subject; and
wherein the PEMF is at a carrier frequency between 300 MHz and 6 GHz.

15. The method of claim 14, wherein the antenna is an external antenna coupled to the transmitter of the mobile telecommunications device.

16. The method of claim 14, wherein the antenna is a near field communication (NFC) antenna.

17. The method of claim 14, wherein the PEMF is emitted as a series of pulses at a pulse frequency of 5 to 100 Hz.
